(19) United States
(12) Patent Application Publication
(10) Pub. No.: US 2017/0328897 A1
(43) Pub. Date: Nov. 16, 2017

(54) METHODS OF PREDICTING RESPONSE TO TREATMENT WITH ANTI-TNFalpha BIOLOGICAL INHIBITORS

(71) Applicant: Fundacio Hospital Universitari Vall D'Hebron- Institut De Recerca, Barcelona (ES)

(72) Inventors: Sara Marsal BARRIL, Barcelona (ES); Antonio Julia CANO, Barcelona (ES)

(21) Appl. No.: 15/421,094

(22) Filed: Jan. 31, 2017

Related U.S. Application Data
(63) Continuation of application No. PCT/IB2015/055840, filed on Jul. 31, 2015.

(30) Foreign Application Priority Data
Aug. 1, 2014 (EP) 14179452.9

(51) Int. Cl. G01N 33/564; A61K 39/00

(52) U.S. Cl. CPC G01N 33/564; A61K 39/0008

(57) ABSTRACT

Provided herein are methods for predicting a response to anti-TNFα biologic treatment in patients suffering from rheumatoid arthritis (RA). The methods can be used to discriminate or categorize patients as likely to respond to a monoclonal antibody-based anti-TNFα biological inhibitor, but not likely to respond to an antibody fragment-based anti-TNFα biological inhibitor or a TNF receptor-based biological inhibitor. Alternatively, the methods are also useful to determine whether patients with RA are likely to respond to an antibody fragment-based anti-TNFα biological inhibitor or a TNF receptor-based biological inhibitor, but not likely to respond to a monoclonal antibody-based anti-TNFα biological inhibitor. Also provided are methods for treating a patient with RA based on the patient's level of rheumatoid factor (RF) and/or anti-cyclic citrullinated peptide autoantibody (ACPA) relative to reference control levels.

### CROSS-REFERENCES TO RELATED APPLICATIONS

This application is a continuation of PCT Application No. PCT/IB2015/055840, filed Jul. 31, 2015, which claims priority to European Application No. 14179452.9, filed Aug. 1, 2014, the disclosures of which are hereby incorporated by reference in their entirety for all purposes.

### BACKGROUND OF THE INVENTION

Rheumatoid arthritis (RA) is a prevalent disease in the Western world. An estimated 0.6% of the population in the United States suffers from it. It is usually treated with Disease Modifying Anti-Rheumatic Drugs (DMARDs), which do not cure the disease but can effectively slow down its progression in a subgroup of patients. The most widely used DMARDs are methotrexate, sulfasalazine, and leflunomide.

The emergence of a new set of biological treatments have proven to be very effective in treating of RA. Clearly, one of the most successful classes of biological therapies include Tumor Necrosis Factor alpha (TNFα) antagonists. TNFα is a pro-inflammatory cytokine that plays a central role in the development of many autoimmune diseases. Currently, the anti-TNFα biological treatments (anti-TNFα biologics) including adalimumab (HUMIRA™), infliximab (REMICADE™), golimumab (SIMPONI™), etanercept (ENBREL™) and certolizumab pegol) (CIMZIA®) have all been approved for the management of RA. The first three (infliximab, adalimumab, golimumab) are monoclonal antibodies with molecular weights over 140 kDa, while the other two (etanercept and certolizumab pegol) are recombinant proteins of significantly smaller size (51 and 91 kDa, respectively). Etanercept is a soluble fusion protein incorporating part of the TNFα receptor, whereas certolizumab pegol is a PEGylated antibody fragment of a monoclonal antibody against TNFα.

Although these biological therapies have proven to be very successful, as nearly half of all treated patients achieve an American College of Rheumatology score of 20% improvement level (ACR20) or even higher, there are limitations to their use. One of the most serious limitations remains unresponsiveness to the drugs. A substantial proportion of treated patients show either partial response or no response to these biological therapies. Since these biologics are costlier compared to DMARDs, there remains a need in the art for novel and effective methods for predicting responsiveness to these anti-TNFα biological therapies.

Autoantibodies such as rheumatoid factor (RF) and anti-cyclic citrullinated peptide (anti-CCP) autoantibodies (ACPAs) have been used as diagnostic markers for RA. Several studies have attempted to correlate the presence or level of RF and/or ACPA to a positive clinical response to one or more anti-TNFα biological inhibitor drug (Potter et al., Ann Rheum Dis, 2009, 68:69-74; Bobbio-Pallavicini et al., Ann Rheum Dis, 2007, 66:302-307; Hyrich et al., Rheumatology, 2006, 45, 1558-1565; Klaasen et al., Rheumatology, 2011, 50:1487-1493). However, these studies have generated conflicting results and have failed to establish a clear association between the level of RF and/or ACPA and a response in patients with RA. As such, there remains a need in the art for a reliable method for determining whether a patient is likely to respond or not respond to a particular anti-TNFα biological inhibitor drug. The present disclosure satisfies this need and provides related advantages as well.

### BRIEF SUMMARY OF THE INVENTION

In a first aspect, provided herein is an in vitro method for predicting whether a subject with rheumatoid arthritis is likely to respond to treatment with an anti-TNFα biological inhibitor. The method includes (a) detecting a level of rheumatoid factor (RF) in a sample obtained from the subject; and (b) determining that the subject is not likely to respond to a monoclonal antibody-based anti-TNFα biological inhibitor, but is likely to respond to an antibody fragment-based anti-TNFα biological inhibitor or a TNF receptor-based biological inhibitor, if the level of RF is higher than an RF reference control level.

In some embodiments, if the level of RF is lower than the RF reference control level, the method further includes (c) detecting a level of anti-cyclic citrullinated peptide autoantibody (ACPA) in a sample obtained from the subject; and (d) determining that the subject is not likely to respond to the antibody fragment-based anti-TNFα biological inhibitor or the TNF receptor-based biological inhibitor, but is likely to respond to the monoclonal antibody-based anti-TNFα biological inhibitor, if the level of ACPA is higher than an ACPA reference control level.

In some embodiments, the sample used to detect the level of RF is the same sample used to detect the level of ACPA, such as a single plasma sample. In other embodiments, the samples used to detect the level of RF and the level of ACPA are different samples such as a plasma sample and a whole blood sample. In other instances, the samples for measuring RF and ACPA levels are different (separate) samples, but of the sample type, such as two plasma samples.

In some embodiments, the method also includes determining that the subject is not likely to respond to the monoclonal antibody-based anti-TNFα biological inhibitor, but is likely to respond to the antibody fragment-based anti-TNFα biological inhibitor or the TNF receptor-based biological inhibitor, if the level of ACPA is lower than the ACPA reference control level.

In a second aspect, provided herein is an in vitro method for predicting whether a subject with rheumatoid arthritis is likely to respond to treatment with an anti-TNFα biological inhibitor. The method includes (a) detecting a level of anti-cyclic citrullinated peptide autoantibody (ACPA) in a sample obtained from the subject; and (b) determining that the subject is likely to respond to a monoclonal antibody-based anti-TNFα biological inhibitor, but is not likely to respond to an antibody fragment-based anti-TNFα biological inhibitor or a TNF receptor-based biological inhibitor, if the level of ACPA is higher than an ACPA reference control level.

In some embodiments, if the level of ACPA is lower than the ACPA reference control level, the subject is not likely to respond to a monoclonal antibody-based anti-TNFα biological inhibitor, but is likely to respond to an antibody fragment-based anti-TNFα biological inhibitor or a TNF receptor-based biological inhibitor.

In some instances, the method further includes (c) detecting a level of rheumatoid factor (RF) in a sample obtained from the subject; and (d) determining that the subject is not likely to respond to a monoclonal antibody-based anti-TNFα biological inhibitor, but is likely to respond to an antibody fragment-based anti-TNFα biological inhibitor or a TNF receptor-based biological inhibitor, if the level of RF is higher than an RF reference control level. Optionally, the method further includes determining that the subject is not likely to respond to the antibody fragment-based anti-TNFα biological inhibitor or the TNF receptor-based biological inhibitor, but is likely to respond to the monoclonal antibody-based anti-TNFα biological inhibitor, if the level of RF is lower than the RF reference control level.

In some embodiments, the monoclonal antibody-based anti-TNFα biological inhibitor of any of the methods disclosed herein is selected from the group consisting of adalimumab (HUMIRA™), infliximab (REMICADE™), golimumab (SIMPONI™), a biosimilar thereof, and a combination thereof.

In some embodiments, the antibody fragment-based anti-TNFα biological inhibitor of any of the methods disclosed herein is selected from the group consisting of certolizumab pegol (CIMZIA™), a biosimilar thereof, and a combination thereof.

In some embodiments, the TNF receptor-based biological inhibitor of any of the methods disclosed herein is selected from the group consisting of etanercept (ENBREL™) pegsunercept, a biosimilar thereof, and a combination thereof.

In some embodiments, the sample of any of the methods disclosed herein is selected from the group consisting of whole blood, plasma, serum, synovial fluid, saliva, and urine.

In some embodiments, the RF reference control level of any of the methods disclosed herein is an RF level within a range of from about 98 IU/mL to about 172 IU/mL. In other embodiments, the RF reference control level is an RF level within a range of from about 107 IU/mL to about 159 IU/mL. In particular embodiments, the RF reference control level is about 148 IU/mL. In yet other embodiments, the RF reference control level corresponds to an RF level within about the 73^{rd} percentile (73%ile) to about the 85^{th} percentile (85%ile) of the RF distribution in a control group, e.g., within about the 75^{th} percentile (75%ile) to about the 83^{rd} percentile (83%ile) or at about the 81^{th} percentile (81%ile).

In some embodiments, the ACPA reference control level of any of the methods disclosed herein is an ACPA level within a range of from about 725 IU/mL to about 754 IU/mL. In particular, embodiments, the ACPA reference control level is about 730 IU/mL. In other embodiments, the ACPA reference control level corresponds to an ACPA level within about the 65^{th} percentile (65%ile) to about the 67^{th} percentile (67%ile) of the ACPA distribution in a control group, e.g., at about the 66^{th} percentile.

In some embodiments, detecting the level of RF and/or the level of ACPA comprises performing an immunoassay. In some instances, the immunoassay comprises an antibody or fragment thereof that specifically binds to RF. In other instances, the immunoassay comprises an antibody or fragment thereof that specifically binds to ACPA.

In a third aspect, provided herein is a method of treating a human subject having rheumatoid arthritis (RA) comprising administering a therapeutically effective amount of a monoclonal antibody-based anti-TNFα biological inhibitor to a human subject suffering from RA and having a level of anti-cyclic citrullinated peptide autoantibody (ACPA) higher than an ACPA reference control level. In some embodiments, the monoclonal antibody-based anti-TNFα biological inhibitor treatment is selected from the group consisting of adalimumab (HUMIRA™) infliximab (REMICADE™), golimumab (SIMPONI®), a biosimilar thereof, and a combination thereof. In some embodiments, the ACPA reference control level is an ACPA level within a range of from about 725 IU/mL to about 754 IU/mL. In particular embodiments, the ACPA reference control level is about 730 IU/mL. In other embodiments, the ACPA reference control level corresponds to an ACPA level within about the 65^{th} percentile (65%ile) to about the 67^{th} percentile (67%ile) of the ACPA distribution in a control group, e.g., at about the 66^{th} percentile.

In a fourth aspect, provided herein is a human subject having rheumatoid arthritis comprising administering a therapeutically effective amount of an antibody fragment-based anti-TNFα biological inhibitor or a TNF receptor-based biological inhibitor to a human subject suffering from RA and having a level of anti-cyclic citrullinated peptide autoantibody (ACPA) lower than an ACPA reference control level. In some embodiments, the human subject has a level of rheumatoid factor (RF) lower than an RF reference control level. In some embodiments, the antibody fragment-based anti-TNFα biological inhibitor is selected from the group consisting of certolizumab pegol (CIMZIA®), a biosimilar thereof, and a combination thereof. In some embodiments, the TNF receptor-based biological inhibitor is selected from the group consisting of etanercept (ENBREL™), pegsunercept, a biosimilar thereof, and a combination thereof. In some embodiments, the ACPA reference control level is an ACPA level within a range of from about 725 IU/mL to about 754 IU/mL. In particular embodiments, the ACPA reference control level is about 730 IU/mL. In other embodiments, the ACPA reference control level corresponds to an ACPA level within about the 65^{th} percentile (65%ile) to about the 67^{th} percentile (67%ile) of the ACPA distribution in a control group, e.g., at about the 66^{th} percentile. In some embodiments, the RF reference control level is an RF level within a range of from about 98 IU/mL to about 172 IU/mL. In other embodiments, the RF reference control level is an RF level within a range of from about 107 IU/mL to about 159 IU/mL. In particular embodiments, the RF reference control level is about 148 IU/mL. In yet other embodiments, the RF reference control level corresponds to an RF level within about the 73^{rd} percentile (73%ile) to about the 85^{th} percentile (85%ile) of the RF distribution in a control group, e.g., within about the 75^{th} percentile (75%ile) to about the 83^{rd} percentile (83%ile) or at about the 81^{th} percentile (81%ile).

In a fifth aspect, provided herein is a method of treating a human subject having rheumatoid arthritis (RA) comprising administering a therapeutically effective amount of an antibody fragment-based anti-TNFα biological inhibitor or a TNF receptor-based biological inhibitor to a human subject suffering from RA and having a level of rheumatoid factor (RF) higher than an RF reference control level. In some instances, the human subject has a level of anti-cyclic citrullinated peptide autoantibody (ACPA) higher than an ACPA reference control level. In some embodiments, the antibody fragment-based anti-TNFα biological inhibitor is selected from the group consisting of certolizumab pegol)(CIMZIA®, a biosimilar thereof, and a combination thereof. In some embodiments, the TNF receptor-based biological inhibitor is selected from the group consisting of etanercept (ENBREL™), pegsunercept, a biosimilar thereof, and a combination thereof. In some embodiments, the RF reference control level is an RF level within a range of from about 98 IU/mL to about 172 IU/mL. In other embodiments, the RF reference control level is an RF level within a range of from about 107 IU/mL to about 159 IU/mL. In particular embodiments, the RF reference control level is about 148 IU/mL. In yet other embodiments, the RF reference control level corresponds to an RF level within about the 73^{rd} percentile (73%ile) to about the 85^{th} percentile (85%ile) of the RF distribution in a control group, e.g., within about the 75^{th} percentile (75%ile) to about the 83^{rd} percentile (83%ile) or at about the 81^{th} percentile (81%ile). In some embodiments, the ACPA reference control level is an ACPA level within a range of from about 725 IU/mL to about 754 IU/mL. In particular embodiments, the ACPA reference control level is about 730 IU/mL. In other embodiments, the ACPA reference control level corresponds to an ACPA level within about the 65^{th} percentile (65%ile) to about the 67^{th} percentile (67%ile) of the ACPA distribution in a control group, e.g., at about the 66^{th} percentile.

In a sixth aspect, provided herein is a method of treating a human subject having rheumatoid arthritis (RA) comprising administering a therapeutically effective amount of a monoclonal antibody-based anti-TNFα biological inhibitor to a human subject suffering from RA and having a level of rheumatoid factor (RF) lower than an RF reference control level and a level of anti-cyclic citrullinated peptide autoantibody (ACPA) higher than an ACPA reference control level. In some embodiments, the RF reference control level is an RF level within a range of from about 98 IU/mL to about 172 IU/mL. In other embodiments, the RF reference control level is an RF level within a range of from about 107 IU/mL to about 159 IU/mL. In particular embodiments, the RF reference control level is about 148 IU/mL. In yet other embodiments, the RF reference control level corresponds to an RF level within about the 73^{rd} percentile (73%ile) to about the 85^{th} percentile (85%ile) of the RF distribution in a control group, e.g., within about the 75^{th} percentile (75%ile) to about the 83^{rd} percentile (83%ile) or at about the 81^{th} percentile (81%ile). In some embodiments, the ACPA reference control level is an ACPA level within a range of from about 725 IU/mL to about 754 IU/mL. In particular embodiments, the ACPA reference control level is about 730 IU/mL. In other embodiments, the ACPA reference control level corresponds to an ACPA level within about the 65^{th} percentile (65%ile) to about the 67^{th} percentile (67%ile) of the ACPA distribution in a control group, e.g., at about the 66^{th} percentile. In some embodiments, the monoclonal antibody-based anti-TNFα biological inhibitor treatment is selected from the group consisting of adalimumab (HUMIRA™), infliximab (REMICADE™), golimumab (SIMPONI™), a biosimilar thereof, and a combination thereof.

In a seventh aspect, provided herein is a method of treating a human subject having rheumatoid arthritis (RA) comprising administering a therapeutically effective amount of an antibody fragment-based anti-TNFα biological inhibitor or a TNF receptor-based biological inhibitor to a human subject suffering from RA and having a level of rheumatoid factor (RF) lower than an RF reference control level and a level of anti-cyclic citrullinated peptide autoantibody (ACPA) lower than an ACPA reference control level. In some embodiments, the RF reference control level is an RF level within a range of from about 98 IU/mL to about 172 IU/mL. In other embodiments, the RF reference control level is an RF level within a range of from about 107 IU/mL to about 159 IU/mL. In particular embodiments, the RF reference control level is about 148 IU/mL. In yet other embodiments, the RF reference control level corresponds to an RF level within about the 73^{rd} percentile (73%ile) to about the 85^{th} percentile (85%ile) of the RF distribution in a control group, e.g., within about the 75^{th} percentile (75%ile) to about the 83^{rd} percentile (83%ile) or at about the 81^{th} percentile (81%ile). In some embodiments, the ACPA reference control level is an ACPA level within a range of from about 725 IU/mL to about 754 IU/mL. In particular embodiments, the ACPA reference control level is about 730 IU/mL. In other embodiments, the ACPA reference control level corresponds to an ACPA level within about the 65^{th} percentile (65%ile) to about the 67^{th} percentile (67%ile) of the ACPA distribution in a control group, e.g., at about the 66^{th} percentile. In some embodiments, the antibody fragment-based anti-TNFα biological inhibitor is selected from the group consisting of certolizumab pegol)(CIMZIA®, a biosimilar thereof, and a combination thereof. In some embodiments, the TNF receptor-based biological inhibitor is selected from the group consisting of etanercept (ENBREL™), pegsunercept, a biosimilar thereof, and a combination thereof.

Disclosed herein are methods for selecting or recommending a type or class of anti-TNFα biological inhibitor for the treatment of RA in a subject in need thereof. The types of anti-TNFα biological inhibitor include a whole monoclonal antibody-based anti-TNFα biological inhibitor, an antibody fragment-based anti-TNFα biological inhibitor or a TNF receptor-based biological inhibitor. The method can be used to distinguish which type of anti-TNFα biological inhibitor should be recommended depending on a subject's level of RF and/or ACPA relative to established cut-off values.

Other objects, features, and advantages of the present invention will be apparent to one of skill in the art from the following detailed description and figures.

### BRIEF DESCRIPTION OF THE DRAWINGS

FIG. 1 shows a graphical representation of the significance values at different anti-CCP (ACPA) thresholds defining high and low titer individuals receiving an anti-TNFα treatment. The x-axis represents autoantibody titers (IU/mL) and the y-axis represents −log 10(P-value). The dotted line shows the titer value where the association is maximal.

FIGS. 2A, 2B and 2C show scanning P-values for anti-CCP thresholds for each anti-TNFα treatment separately. FIG. 2A represents patients receiving infliximab (IFX). FIG. 2B represents patients receiving etanercept (ETN). FIG. 2C represents patients receiving adalimumab (ADA). In all graphs, the x axis represent autoantibody titers (IU/mL) and the y-axis represents −log 10(P-value). The dotted line shows the titer value where the association is maximal.

FIG. 3 shows scanning P-values for rheumatoid factor (RF) thresholds for all patients in the study described herein. The x-axis represents autoantibody titers (IU/mL) and the y-axis represents −log 10(P-value). The dotted line shows the titer value where the association is maximal.

FIGS. 4A, 4B and 4C show scanning P-values for RF thresholds for each anti-TNF α treatment separately. FIG. 4A represents patients receiving infliximab (IFX). FIG. 4B represents patients receiving etanercept (ETN). FIG. 4C represents patients receiving adalimumab (ADA). In all graphs, the x-axis represent autoantibody titers (IU/mL) and the y-axis represents −log 10(P-value). The dotted line shows the titer value where the association is maximal.

FIG. 5 shows scanning P-values for rheumatoid factor (RF) thresholds for patients receiving a monoclonal antibody-based anti-TNF α biological inhibitor. The x-axis represents autoantibody titers (IU/mL) and the y-axis represents −log 10(P-value). The dotted line shows the titer value where the association is maximal.

### DETAILED DESCRIPTION OF THE INVENTION

### I. INTRODUCTION

The methods provided herein are based, in part, on the surprising discovery of novel cut-off levels (threshold values) for rheumatoid factor (RF) and anti-cyclic citrullinated peptide autoantibody (ACPA) that can be used to predict whether a patient with rheumatoid arthritis (RA) is likely or unlikely to respond to a specific type of anti-TNFα biological inhibitor, such as a whole monoclonal antibody-based anti-TNFα biological inhibitor (e.g., adalimumab (HUMIRA™), infliximab (REMICADE™) and golimumab (SIMPONI®)), an antibody fragment-based anti-TNFα biological inhibitor (e.g., certolizumab pegol))(CIMZIA®)) or a TNF receptor-based biological inhibitor (e.g., etanercept (ENBREL™) and pegsunercept). In particular, a single cut-off level for RF and/or a single cut-off level for ACPA can accurately determine, discriminate between, and/or stratify which of the three types of anti-TNFα biological inhibitors should be selected or recommended for the treatment of RA and/or administered to a patient for the treatment of RA.

In certain aspects, the methods disclosed herein utilize a threshold or reference level within a standard RF positive range or within a standard RF positive and negative range to predict or select responders vs. non-responders for a particular class or type of anti-TNFα biological inhibitor. In certain other aspects, the methods disclosed herein utilize a threshold or reference level within a standard ACPA positive range or within a standard ACPA positive and negative range to predict or select responders vs. non-responders for a particular class or type of anti-TNFα biological inhibitor. The methods include measuring the level of RF alone, the level of ACPA alone, or the level of RF and the level of ACPA to predict positive response or non-response to a monoclonal antibody-based anti-TNFα biological inhibitor, antibody fragment-based anti-TNFα biological inhibitor or TNF receptor-based biological inhibitor.

### II. DEFINITIONS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “rheumatoid arthritis” or “RA” includes an autoimmune disease that causes chronic inflammation of the connective tissues in the body, most particularly, the joints and the tissue around the joints. In rheumatoid arthritis, multiple joints are usually inflamed in a symmetrical pattern (e.g., both sides of the body are affected). The term “rheumatoid arthritis” also includes conditions such as Sjogren's syndrome, where the inflammation affects organs and areas of the body other than the joints, e.g., the glands of the eyes and mouth, causing dryness of these areas.

The term “anti-TNFα biological inhibitor” refers to a biologic-based anti-TNFα drug or TNFα blocking agent including whole monoclonal antibodies such as infliximab, adalimumab, and golimumab; antibody fragments such as certolizumab pegol; fusion proteins such as Ig fusion proteins or Fc fusion proteins (e.g., TNF receptor fusion proteins including etanercept) similar naturally- or non-naturally-occurring molecules, and/or recombinant and/or engineered forms thereof, that, directly or indirectly, inhibit TNFα activity, such as by inhibiting interaction of TNFα with a cell surface receptor for TNFα, inhibiting TNFα protein production, inhibiting TNFα secretion from cells, inhibiting TNFα receptor signaling or any other means resulting in decreased TNFα activity in a subject.

As used herein, the term “monoclonal antibody-based biological inhibitor” refers to a biological inhibitor or antagonist consisting of whole monoclonal antibody, such as a monoclonal antibody having two heavy chains and two light chains joined together to form a “Y” shaped molecule. The two heavy chains are linked together by disulfide bonds and each heavy chain is linked to a light chain by a disulfide bond. Such a monoclonal antibody-based biological inhibitor may include a chimeric monoclonal antibody

As used herein, the term “monoclonal antibody” or “whole monoclonal antibody” which can be used interchangeably does not include a single domain antibody, or an antibody fragment such as a Fab fragment, Fab′ fragment, other antigen binding fragment, Fc fragment, F(ab′)₂ fragment, single chain variable fragment (scFv), variants thereof, or derivatives thereof (e.g., conjugated antibody fragments). The term “monoclonal antibody” does not include diabodies composed of dimers of scFvs (di-scFv), tandem scFvs, miniaturized monoclonal antibodies, unibodies, nanobodies, or minibodies. Non-limiting examples of a monoclonal antibody-based anti-TNFα biological inhibitors for use to treat rheumatoid arthritis include adalimumab (ADA; HUMIRA™), infliximab (IFX; REMICADE™), and golimumab (GOL; S IMPONI®.

As used herein, the term “antibody fragment-based biological inhibitor” refers to a biological inhibitor or antagonist consisting of an antibody fragment such as a Fab fragment, Fab′ fragment, other antigen binding fragment, Fc fragment, F(ab′)₂ fragment, single chain variable fragment (scFv), variants thereof, or derivatives thereof (e.g., conjugated antibody fragments). The term includes diabodies, multimeric scFvs, bis-scFv, triabodies, tetrabodies, miniaturized monoclonal antibodies, unibodies, nanobodies, or minibodies. Non-limiting examples of an antibody fragment-based anti-TNFα biological inhibitor include certolizumab pegol, CDP870, or another anti-TNFα antibody fragment, variant thereof, derivative thereof, and conjugates thereof. In some instances, the antibody-fragment is modified such as covalently conjugated to a linear or branched poly(ethylene glycol) (PEG) molecule or other polymers such as polysialic acid, HPMA, dextran, albumin, and the like. Certolizumab pegol is a PEGylated Fab fragment of a humanized monoclonal antibody that specifically binds to TNFα.

As used herein, the term “TNF receptor-based biological inhibitor” refers to a biological inhibitor or antagonist consisting of the p75 tumor necrosis factor receptor or a portion thereof such as the extracellular domain of the TNF receptor. A non-limiting example of a TNF receptor-based biological inhibitor includes etanercept (ETN; ENBREL™) which is a dimeric fusion protein consisting of the extracellular domain (soluble domain) of the TNF receptor linked to the Fc domain of IgG1, and pegsunercept.

The term “likely to respond” in the context of a therapy refers to an above-average likelihood (chance or probability) that an individual will have a positive response to a treatment.

The term “not likely to respond” in the context of therapy refers to refers to an above-average likelihood (chance or probability) that an individual will not have a positive response to a treatment.

The term “positive response” with respect to a therapeutic treatment refers to at least a partial marked reduction in the severity, an amelioration of one or more symptoms of a patient's disease or disorder, or slows disease progression. A positive clinical response to such a treatment can be measured by applying the American College of Rheumatology/European League Against Rheumatism (ACR/EULAR) RA classification criteria for RA (Aletaha et al., Arth Rheum, 2010, 62(9):2569-2581). The criteria classifies an individual as a non-, moderate and good responder depending on the degree of joint involvement, the presence or level of serological markers, the presence or level of acute phase proteins, and the duration of the individual's symptoms.

The term “treat,” “treating” or “treatment” refers to an action that reduces the severity or symptoms of the disease or disorder, or retards or slows the progression or symptoms of the disease or disorder in a patient is suffering from the specified disease or disorder.

The term “therapeutically effective amount or dose” includes a dose of a drug (e.g., a PDE4 inhibitor) that is capable of achieving a therapeutic effect in a subject in need thereof. For example, a therapeutically effective amount of a drug useful for treating RA or PsA can be the amount that is capable of preventing or relieving one or more symptoms associated with RA or PsA. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “rheumatoid factor” or “RF” refers to an autoantibody (i.e., an antibody directed against an organism's own tissues) that is typically directed against (i.e., binds to) the Fc (fragment crystallizable) portion of immunoglobulin G (IgG). Rheumatoid factor is most often an IgM autoantibody, but may also be an IgG, IgA, IgD, or IgE autoantibody. These autoantibodies are often found to be elevated in rheumatoid arthritis patients, but can also be abnormally high in other pathologies such as viral infection, hepatitis, systemic lupus, erythematosus or organ rejection. Sometimes it can even be elevated in healthy individuals. Therefore, although the presence of RF can be an indication of rheumatoid arthritis, it can be caused by other pathologies, and conversely, its absence cannot be used to rule out RA.

The term “anti-cyclic citrullinated peptide autoantibody,” “anti-citrullinated peptide antibody,” or “ACPA” includes an autoantibody that specifically targets one or more epitopes in a peptide, polypeptide, or protein sequence where one or more arginine residues have been converted by the enzyme peptidylarginine deiminase into a citrulline residue during a post-translational modification. The presence or level of anti-citrullinated protein antibodies can be detected, determined, or measured using natural or synthetic citrullinated peptides which are immunologically reactive (i.e., immunoreactive) with such antibodies. Non-limiting examples of synthetic citrullinated peptides include cyclic citrullinated peptides (CCP) such as CCP1, which contains a single cyclic citrullinated peptide derived from filaggrin, and/or CCP2, which is a combination of citrullinated peptides selected from screening libraries of citrullinated peptides. Assays for detecting anti-CCP antibodies are available from INOVA Diagnostics, Euro-Diagnostica, Axis-Shield, Phadia, Orgentec Diagostika, and Abbott Diagnostics. Anti-citrullinated protein antibodies are autoantibodies typically associated with rheumatoid arthritis.

The term “cut-off level,” “threshold level” or “reference control level” refers to a predefined number or value for a given marker on the basis of population analysis. A cut-off level is used for comparison to a value of a test subject. Thus, the cut-off level is based on analysis of index values determined using statistical analysis or an algorithm. Those of skill in the art will recognize that a cut-off level can be determined according to the needs of the user and characteristics of the analyzed population. Once a cut-off level is determined, it is compared to an index value or measured value for an individual.

The term “ACPA reference control level” refers to a cut-off value for the level of anti-cyclic citrullinated peptide autoantibody (ACPA) detected in a biological sample taken from a control group, such as a population of subject who have RA, and in some instances were administered a specific anti-TNFα biological inhibitor. The term “RF reference control level” refers to a cut-off value for the level of rheumatoid factor (RF) detected in a biological sample taken from a subject control group, such as a population of subject who have RA, and in some instances were administered a specific anti-TNFα biological inhibitor.

The term “percentile of RF distribution in a control group” refers to a percentile distribution of the level of rheumatoid factor (RF) in a control population such as a group of RA patients. In some cases, the RA patients are administered a specific anti-TNFα biological inhibitor.

The term “percentile of ACPA distribution in a control group” refers to a percentile distribution of the level of anti-CCP autoantibody (ACPA) in a control population such as a group of RA patients. In some cases, the RA patients are administered a specific anti-TNFα biological inhibitor.

The term “immunoassay” refers to a immunological binding assay that uses an antibody or a fragment thereof that specifically binds to a protein, peptide or antigen of choice, or an antigenic subsequence thereof.

The term “specifically binds to” refers to “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background.

The term “biosimilar” refers to a pharmaceutical agent or drug that is approved for clinical use based on evidence that it is similar, e.g., structurally and functionally similar, to a reference, approved pharmaceutical agent or drug and has not clinically meaningful differences in safety and effectiveness compared to the reference agent or drug.

The term “sample” refers to any biological specimen obtained from an individual. Suitable samples for use in the present invention include, without limitation, whole blood, plasma, serum, synovial fluid, saliva, urine, stool, tears, any other bodily fluid, tissue samples (e.g., biopsy), and cellular extracts thereof (e.g., red blood cellular extract). In a preferred embodiment, the sample is a whole blood, plasma or serum sample. The use of samples such as serum, saliva, and urine is well known in the art (see, e.g., Hashida et al., J. Clin. Lab. Anal., 11:267-86 (1997)). One skilled in the art will appreciate that samples such as serum samples can be diluted prior to the analysis of marker levels. The term “bodily fluid” refers to a liquid originating from inside the living body. It includes fluids that are excreted or secreted from the body as well as those that normally are not.

The term “subject,” “patient,” or “individual” typically includes humans, but can also include other animals such as, e.g., other primates, rodents, canines, felines, equines, ovines, porcines, and the like. In some embodiments, the term “subject” refers to a human subject suffering from rheumatoid arthritis and in need of treatment.

The term “antibody or fragment thereof that specifically binds to rheumatoid factor” refers to an immunoglobulin or a fragment thereof that is able to selectively bind RF. It includes monoclonal and polyclonal antibodies, and any part of an antibody having the size and conformation suitable to bind an epitope of RF. Suitable fragments include F(ab), F(ab′), nanobodies and Fv. An “epitope” is the part of an antigen that is recognized by an individual's immune system (B-cells, T-cells or antibodies).

The term “antibody or fragment thereof that specifically binds to ACPA” refers to an immunoglobulin or a fragment thereof that is able to selectively bind ACPA. It includes monoclonal and polyclonal antibodies, and any part of an antibody having the size and conformation suitable to bind an epitope of ACPA. Suitable fragments include F(ab), F(ab′), nanobodies and Fv. An “epitope” is the part of an antigen that is recognized by an individual's immune system (B-cells, T-cells or antibodies).

### III. DETAILED DESCRIPTION OF THE EMBODIMENTS

A. Treatment Selection for Patients Suffering from Rheumatoid Arthritis

Provided herein is a method for selecting a subject, e.g., a human subject suffering from RA, who should receive an anti-TNFα biological inhibitor. Also provided is a method for predicting whether the subject is likely or not likely to have a positive clinical response to such a treatment. In addition, disclosed herein is a method for selecting or recommending a type of anti-TNFα biological inhibitor for the treatment of RA in a subject in need thereof. The method is based, in part, on an association between the level of rheumatoid factor and/or anti-cyclic citrullinated peptide autoantibodies relative to reference control levels and a likelihood of response to a monoclonal antibody-based anti-TNFα biological inhibitor (e.g., adalimumab, infliximab and golimumab), antibody fragment-based anti-TNFα biological inhibitor (e.g., certolizumab pegol), or a TNF receptor-based biological inhibitor (e.g., etanercept or pegsunercept).

In some embodiments, an RF level higher than an RF reference control level indicates that the subject with RA is not likely to respond or is likely to have a poor response or non-response to a monoclonal antibody-based anti-TNFα biological inhibitor. A higher RF level relative to an RF reference control level can also indicate that the subject is likely to respond or have a positive response to an antibody fragment-based anti-TNFα biological inhibitor or a TNF receptor-based biological inhibitor. As such, a patient with RA and a high RF level can be administered an antibody fragment-based anti-TNFα biological inhibitor or a TNF receptor-based biological inhibitor, and not a monoclonal antibody-based anti-TNFα biological inhibitor.

In other embodiments, a subject with RA having an RF level that is lower than an RF reference control level and having an ACPA level higher than an ACPA reference control level is likely to respond to a monoclonal antibody-based anti-TNFα biological inhibitor. Such a subject is also not likely to respond to an antibody fragment-based anti-TNFα biological inhibitor or a TNF receptor-based biological inhibitor. In some embodiments, a monoclonal antibody-based anti-TNFα biological inhibitor is selected or recommended for a subject with RA and having an RF level that is lower than an RF reference control level and having an ACPA level higher than an ACPA reference control level. In some cases, a subject with RA and a low RF level and high ACPA level is administered a monoclonal antibody-based anti-TNFα biological inhibitor.

In yet other embodiments, a subject with RA who has an RF level that is lower than the RF reference control level and an ACPA level lower than the ACPA reference control level is likely to respond to an antibody fragment-based anti-TNFα biological inhibitor or a TNF receptor-based biological inhibitor, but is not likely to respond to monoclonal antibody-based anti-TNFα biological inhibitor. In some embodiments, an antibody fragment-based anti-TNFα biological inhibitor is selected or recommended for a subject suffering from RA and having an RF level that is lower than the RF reference control level and an ACPA level lower than the ACPA reference control level. In other embodiments, a TNF receptor-based biological inhibitor is selected or recommended for a subject suffering from RA and having an RF level that is lower than the RF reference control level and an ACPA level lower than the ACPA reference control level. In some instances, a patient with RA and a low RF level and low ACPA level is administered an antibody fragment-based anti-TNFα biological inhibitor or a TNF receptor-based biological inhibitor, and not a monoclonal antibody-based anti-TNFα biological inhibitor.

B. Detecting Rheumatoid Factor (RF) Levels

The methods disclosed herein can include determining the level of rheumatoid factor (RF) in a sample from a patient suffering from RA. Rheumatoid factors (RFs) are autoantibodies found in every immunoglobulin subclass (IgE, IgM, IgA, IgD and IgG) and directed to the constant Fc-region of immunoglobulins of the IgG subclass. During the earliest stages of rheumatoid arthritis some patients (about 15-20% of patients) do not have detectable levels of RF. In some cases, patients with detectable RF do not have rheumatoid arthritis. The presence or level of RF can be determined by agglutination assays, laser and rate nephelometry, enzyme-linked immunoassays (EIAs) or enzyme linked immunosorbent assays (ELISAs). Commercial systems, kits, and services for measuring RF in a sample are available from, e.g., Roche Diagnostic, Euro-Diagnostica, Phadia, Abbott Diagnostics, ImmunoLab, Abnova, Beckman Coulter, Thermo Fisher Scientific, INOVA Diagnostics, Siemens Healthcare Diagnostics Inc., and Quest Diagnostics.

RF levels can be measured in a biological sample, e.g., whole blood, plasma, serum, synovial fluid, saliva, urine, tears, sweat or another bodily fluid obtained from a patient. The sample used to determine the level of RF can be the same sample or a different sample than the one used to measure the level of ACPA. As an illustrative example, the sample used to measure RF may be serum and the sample used to measure ACPA may be whole blood. Alternatively, a single sample can be used for detecting RF level and ACPA level. In other cases, two samples of the sample type are used for detecting RF and ACPA levels.

If the level, amount, concentration or titer of RF in a sample from the test subject having RA is higher (e.g., 1.01-fold, 1.05-fold, 1.10-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 30-fold, 40-fold, 50-fold, 100-fold or more higher) than a cut-off value, this would indicate, e.g., a likelihood of a positive clinical response to an antibody fragment-based anti-TNFα biological inhibitor or a TNF receptor-based biological inhibitor. In some embodiments, the level of RF is deemed to be “higher” if it is at least 1, 2, 3, 4, 5, 10, 15, 20 or more standard deviations greater than the RF reference control level.

If a level, amount or concentration in a sample from the test subject is significantly lower (e.g., 1.01-fold, 1.05-fold, 1.10-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 30-fold, 40-fold, 50-fold, 100-fold or more lower) than the threshold value, this may indicate that the level of RF can be measured to determine the subject's response to an anti-TNFα biological inhibitor. In some embodiments, the level of RF is deemed to be “lower” if it is at least 1, 2, 3, 4, 5, 10, 15, 20, or more standard deviations lower than the RF reference control level.

In some embodiments, the cut-off level for RF or the RF reference control level is about 148 IU/mL. In other embodiments, the cut-off level for RF or the RF reference control level is an RF level within a range of from about 107 IU/mL to about 159 IU/mL, e.g., about 107 IU/mL, 109 IU/mL, 110 IU/mL, 111 IU/mL, 113 IU/mL, 115 IU/mL, 117 IU/mL, 119 IU/mL, 120 IU/mL, 121 IU/mL, 123 IU/mL, 125 IU/mL, 127 IU/mL, 129 IU/mL, 130 IU/mL, 131 IU/mL, 133 IU/mL, 135 IU/mL, 137 IU/mL, 139 IU/mL, 140 IU/mL, 141 IU/mL, 143 IU/mL, 145 IU/mL, 147 IU/mL, 149 IU/mL, 150 IU/mL, 151 IU/mL, 153 IU/mL, 155 IU/mL, 157 IU/mL, or 159 IU/mL. In other embodiments, the cut-off level for RF or the RF reference control level is an RF level within a range of from about 107 IU/mL to about 159 IU/mL, e.g., about 107 IU/mL to about 159 IU/mL, 110 IU/mL to about 159 IU/mL, 115 IU/mL to about 159 IU/mL, 120 IU/mL to about 159 IU/mL, 123 IU/mL to about 159 IU/mL, 130 IU/mL to about 159 IU/mL, 135 IU/mL to about 159 IU/mL, 140 IU/mL to about 159 IU/mL, 145 IU/mL to about 159 IU/mL, 150 IU/mL to about 159 IU/mL, 107 IU/mL to about 150 IU/mL, 107 IU/mL to about 145 IU/mL, 107 IU/mL to about 140 IU/mL, 107 IU/mL to about 140 IU/mL, 107 IU/mL to about 135 IU/mL, 107 IU/mL to about 130 IU/mL, 107 IU/mL to about 125 IU/mL, 107 IU/mL to about 120 IU/mL, 107 IU/mL to about 115 IU/mL, or 107 IU/mL to about 110 IU/mL.

In some embodiments, the cut-off level for RF or the RF reference control level is an RF level within a range of from about 98 IU/mL to about 172 IU/mL, e.g., about 98 IU/mL, 100 IU/mL, 102 IU/mL, 104 IU/mL, 106 IU/mL, 108 IU/mL, 110 IU/mL, 112 IU/mL, 114 IU/mL, 116 IU/mL, 118 IU/mL, 120 IU/mL, 122 IU/mL, 124 IU/mL, 126 IU/mL, 128 IU/mL, 130 IU/mL, 132 IU/mL, 134 IU/mL, 136 IU/mL, 138 IU/mL, 140 IU/mL, 142 IU/mL, 144 IU/mL, 146 IU/mL, 148 IU/mL, 150 IU/mL, 152 IU/mL, 154 IU/mL, 156 IU/mL, 158 IU/mL, 160 IU/mL, 162 IU/mL, 164 IU/mL, 166 IU/mL, 168 IU/mL, 170 IU/mL, or 172 IU/mL. In other embodiments, the cut-off level for RF or the RF reference control level is an RF level within a range of from about 98 IU/mL to about 172 IU/mL, e.g., about 98 IU/mL to about 172 IU/mL, about 100 IU/mL to about 172 IU/mL, about 105 IU/mL to about 172 IU/mL, about 110 IU/mL to about 172 IU/mL, about 115 IU/mL to about 172 IU/mL, about 120 IU/mL to about 172 IU/mL, about 125 IU/mL to about 172 IU/mL, about 130 IU/mL to about 172 IU/mL, about 135 IU/mL to about 172 IU/mL, about 140 IU/mL to about 172 IU/mL, about 145 IU/mL to about 172 IU/mL, about 150 IU/mL to about 172 IU/mL, about 155 IU/mL to about 172 IU/mL, about 160 IU/mL to about 172 IU/mL, about 165 IU/mL to about 172 IU/mL, about 170 IU/mL to about 172 IU/mL, about 98 IU/mL to about 170 IU/mL, about 98 IU/mL to about 165 IU/mL, about 98 IU/mL to about 160 IU/mL, about 98 IU/mL to about 155 IU/mL, about 98 IU/mL to about 150 IU/mL, about 98 IU/mL to about 145 IU/mL, about 98 IU/mL to about 140 IU/mL, about 98 IU/mL to about 135 IU/mL, about 98 IU/mL to about 130 IU/mL, about 98 IU/mL to about 125 IU/mL, about 98 IU/mL to about 120 IU/mL, about 98 IU/mL to about 115 IU/mL, about 98 IU/mL to about 110 IU/mL, about 98 IU/mL to about 105 IU/mL, or about 98 IU/mL to about 100 IU/mL.

In some embodiments, a quantile measurement of the level of RF within a given population, such as a group of RA patients receiving a specific anti-TNFα blocking agent is generated. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of levels (e.g., tertiles, quartile, quintiles, etc.), or their cumulative indices (e.g., quartile sums of marker levels to obtain quartile sum scores (QSS), etc.) as variables in the statistical analyses (just as with continuous variables).

In some embodiments, the cut-off value for RF or the RF reference control level determined for a patient's response to an anti-TNFα biological inhibitor corresponds to about the 81^{st} percentile (81%ile) of RF distribution in RA patients including RA patients receiving an anti-TNFα biological inhibitor.

In other embodiments, the cut-off value for RF or the RF reference control level is an RF level within a range of from about the 75^{th} to about the 83^{rd} percentile, e.g., about the 75^{th}, 76^{th}, 77^{th}, 78^{th}, 79^{th}, 80^{th}, 81^{st}, 82^{nd} or 83^{rd} percentile (i.e., about the 75%ile, 76%ile, 77%ile, 78%ile, 79%ile, 80%ile 81%ile 82%ile, or 83%ile) of RF distribution in RA patients, e.g., RA patients receiving an anti-TNFα biological inhibitor. In some embodiments, the cut-off value for RF or the RF reference control level is an RF level within a range of from about the 75^{th} to about the 83^{rd} percentile, e.g., about the 75^{th} to about the 83^{rd}, about the 76^{th} to about the 83^{rd}, about the 77^{th} to about the 83^{rd}, about the 78^{th} to about the 83^{rd}, about the 79^{th} to about the 83^{rd}, about the 80^{th} to about the 83^{rd}, about the 81^{st} to about the 83^{rd}, about the 82^{nd} to about the 83^{rd}, about the 75^{th} to about the 82^{nd}, about the 75^{th} to about the 81^{st}, about the 75^{th} to about the 80^{th}, about the 75^{th} to about the 79^{th}, about the 75^{th} to about the 78^{th}, about the 75^{th} to about the 77^{th}, or about the 75^{th} to about the 76^{th} percentile of RF distribution in RA patients, e.g., RA patients receiving an anti-TNFα biological inhibitor.

In some embodiments, the cut-off value for RF or the RF reference control level is an RF level within a range of from about the 73^{rd} to about the 85^{th} percentile, e.g., about the 73^{rd}, 74^{th}, 75^{th}, 76^{th}, 77^{th}, 78^{th}, 79^{th}, 80^{th}, 81^{st}, 82^{nd}, 83rd, 84^{th}, or 85^{th} percentile (i.e., about the 73%ile, 74%ile, 75%ile, 76%ile, 77%ile, 78%ile, 79%ile, 80%ile 81%ile 82%ile, 83%ile, 84%ile, or 85%ile) of RF distribution in RA patients, e.g., RA patients receiving an anti-TNFα biological inhibitor. In other embodiments, the cut-off value for RF or the RF reference control level is an RF level within a range of from about the 73^{rd} to about the 85^{th} percentile, e.g., about the 73^{rd} to about the 85^{th}, about the 74^{th} to about the 85^{th}, about the 75^{th} to about the 85^{th}, about the 76^{th} to about the 85^{th}, about the 77^{th} to about the 85^{th}, about the 78^{th} to about the 85^{th}, about the 79^{th} to about the 85^{th}, about the 80^{th} to about the 85^{th}, about the 81^{st} to about the 85^{th}, about the 82^{nd} to about the 85^{th}, about the 83^{rd} to about the 85^{th}, about the 84^{th} to about the 85^{th} about the 73^{rd} to about the 85^{th}, about the 73^{rd} to about the 84^{th}, about the 73^{rd} to about the 83^{rd} about the 73^{rd} to about the 82^{nd}, about the 73^{rd} to about the 81^{st}, about the 73^{rd} to about the 80^{th}, about the 73^{rd} to about the 79^{th}, about the 73^{rd} to about the 78^{th}, about the 73^{rd} to about the 77^{th}, about the 73^{rd} to about the 76^{th}, about the 73^{rd} to about the 75^{th}, or about the 73^{rd} to about the 74^{th} percentile of RF distribution in RA patients, e.g., RA patients receiving an anti-TNFα biological inhibitor.

C. Detecting Anti-Cyclic Citrullinated Peptide Autoantibody (ACPA) Levels

The method disclosed herein can include determining the level of anti-cyclic citrullinated peptide autoantibody (ACPA) in a sample from a patient suffering from RA. Citrullinated antigens (e.g., citrullinated filaggrin, citrullinated vimentin, citrullinated fibrinogen, citrullinated lamin B 1, citrullinated enolase, citrullinated intermediate filament-derived peptides, viral citrullinated peptides, and fragments thereof) have been shown to be reactive with rheumatoid arthritis autoantibodies in 76% of rheumatoid arthritis sera, with a specificity of 96%. Immunoassays based on the detection of autoantibodies to cyclic citrullinated peptides (anti-cyclic citrullinated peptide autoantibodies; ACPAs) are also useful for the present invention. Examples of such assays include, but are not limited to, the IMMULITE 2000™ anti-CCP IgG assay (Siemens Healthcare Diagnostics, Inc., Tarrytown, N.Y.), the anti-CCP EDIA™ (Euro-Diagnostica, Malmo, Sweden), and the anti-CCP ELISA (Axis-Shield, Dundee, Scotland). Additional assays for the detection of anti-citrullinated peptide antibodies include the first, second, and third generation anti-citrullinated peptides assays, which are also referred to as the anti-CCP1 assay, the anti-CCP2 assay, and the anti-CCP3 assay, respectively. Other commercial systems, kits, and services for measuring ACPA in a sample are available from, e.g., Roche Diagnostic, Euro-Diagnostica, Axis-Shield, Phadia, Beckman Coulter, Thermo Fisher Scientific, INOVA Diagnostics, Abbott Diagnostics, Siemens Healthcare Diagnostics Inc., and Quest Diagnostics.

If the level, amount, concentration or titer of ACPA in a sample from the test subject having RA is higher (e.g., 1.01-fold, 1.05-fold, 1.10-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 30-fold, 40-fold, 50-fold, 100-fold or more higher) than the threshold value, this would indicate, e.g., a likelihood of a positive clinical response to a monoclonal antibody-based anti-TNFα biological inhibitor, but not to have a positive clinical response to an antibody fragment-based anti-TNFα biological inhibitor or a TNF receptor-based biological inhibitor. In some embodiments, the level of ACPA is deemed to be “higher” if it is at least 1, 2, 3, 4, 5, 10, 15, 20, or more standard deviations greater than the ACPA reference control level.

If a level, amount or concentration in a sample from the test subject is significantly lower (e.g., 1.01-fold, 1.05-fold, 1.10-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 30-fold, 40-fold, 50-fold, 100-fold or more lower) than a cut-off value, this would indicate, e.g., a likelihood of a positive clinical response to an antibody fragment-based anti-TNFα biological inhibitor or a TNF receptor-based biological inhibitor, but is not likely to positively respond to a monoclonal antibody based anti-TNFα biological inhibitor or a TNF receptor-based biological inhibitor. In some embodiments, the level of ACPA is deemed to be “lower” if it is at least 1, 2, 3, 4, 5, 10, 15, 20 or more standard deviations lower than the ACPA reference control level.

In some embodiments, the cut-off level for ACPA or the ACPA reference control level is about 730 IU/mL. In other embodiments, the cut-off level for ACPA or the ACPA reference control level is an ACPA level within a range of from about 725 IU/mL to about 754 IU/mL, e.g., about 725 IU/mL, 726 IU/mL, 727 IU/mL, 728 IU/mL, 729 IU/mL, 730 IU/mL, 731 IU/mL, 732 IU/mL, 733 IU/mL, 734 IU/mL, 735 IU/mL, 736 IU/mL, 737 IU/mL, 738 IU/mL, 739 IU/mL, 740 IU/mL, 741 IU/mL, 742 IU/mL, 743 IU/mL, 744 IU/mL, 745 IU/mL, 746 IU/mL, 747 IU/mL, 748 IU/mL, 749 IU/mL, 750 IU/mL, 751 IU/mL, 752 IU/mL, 753 IU/mL, or 754 IU/mL. In yet other embodiments, the cut-off level for ACPA or the ACPA reference control level is an ACPA level within a range of from about 725 IU/mL to about 754 IU/mL, e.g., about 725 IU/mL to about 754 IU/mL, about 725 IU/mL to about 750 IU/mL, about 725 IU/mL to about 745 IU/mL, about 725 IU/mL to about 740 IU/mL, about 725 IU/mL to about 735 IU/mL, about 725 IU/mL to about 730 IU/mL, about 730 IU/mL to about 754 IU/mL, about 735 IU/mL to about 754 IU/mL, about 740 IU/mL to about 754 IU/mL, about 745 IU/mL to about 754 IU/mL, or about 750 IU/mL to about 754 IU/mL.

In some embodiments, a quantile measurement of the level of ACPA within a given population, such as a group of RA patients receiving a specific anti-TNFα blocking agent is generated. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of levels (e.g., tertiles, quartile, quintiles, etc.), or their cumulative indices (e.g., quartile sums of marker levels to obtain quartile sum scores (QSS), etc.) as variables in the statistical analyses (just as with continuous variables).

In some embodiments, the cut-off value for ACPA or the ACPA reference control level determined for a patient's response to an anti-TNFα biological inhibitor corresponds to the 66^{th} percentile (66%ile) of ACPA distribution in RA patients including RA patients receiving an anti-TNFα biological inhibitor.

In other embodiments, the cut-off value for ACPA or the ACPA reference control level is an ACPA level within a range of from about the 65.8^{th} to about the 66.9^{th} percentile, e.g., about the 65.8^{th}, 65.9^{th}, 66.0, 66.0, 66.1^{st}, 66.2^{nd}, 66.3^{rd}, 66.4^{th}, 66.5^{th}, 66.6^{th}, 66.7^{th}, 66.8^{th}, or 66.9^{th} percentile (i.e., about the 65.8%ile, 65.9%ile, 66.0%ile, 66.1%ile, 66.2%ile, 66.3%ile, 66.4%ile, 66.5%ile, 66.6%ile, 66.7%ile, 66.8%ile, or 66.9%ile) of ACPA distribution in RA patients, e.g., RA patients receiving an anti-TNFα biological inhibitor. In other embodiments, the cut-off value for ACPA or the ACPA reference control level is an ACPA level within a range of from about the 65.8^{th} to about the 66.9^{th} percentile, e.g., about the 65.8^{th} to about the 66.9^{th}, about the 65.8^{th} to about the 66.7^{th}, about the 65.8^{th} to about the 66.5^{th}, about the 65.8^{th} to about the 66.3^{rd}, about the 65.8^{th} to about the 66.1⁴, about the 65.8^{th} to about the 66.0, about the 65.9^{th} to about the 66.9^{th}, about the 66.0 to about the 66.9^{th}, about the 66.1^{st} to about the 66.9^{th}, about the 66.2^{nd} to about the 66.9^{th}, about the 66.3^{rd} to about the 66.9^{th}, about the 66.4^{th} to about the 66.9^{th}, about the 66.5^{th} to about the 66.9^{th}, about the 66.6^{th} to about the 66.9^{th}, about the 66.7^{th} to about the 66.9^{th}, or about the 66.8^{th} to about the 66.9^{th} percentile of ACPA distribution in RA patients, e.g., RA patients receiving an anti-TNFα biological inhibitor.

D. Establishing Reference Control Levels for RF and ACPA

In order to establish a reference control level (cut-off level or threshold value) for either RF or ACPA, a reference population of subjects can be used. In some embodiments, a population of RA patients receiving an anti-TNFα biological inhibitor is used. In other embodiments, the control or reference population includes subjects with RA who are receiving a monoclonal antibody-based anti-TNFα biological inhibitor. In another embodiment, the control or reference population includes subjects with RA who are receiving an antibody fragment-based anti-TNFα biological inhibitor. In yet other embodiments, the control or reference population includes subjects with RA who are receiving a TNF receptor-based biological inhibitor. In some cases, the reference population includes a plurality of responders and non-responders to a particular anti-TNFα biological inhibitor.

These reference subjects can be within the appropriate parameters, if applicable, for the purpose of therapy selection for treating RA using the methods of the present invention. Optionally, the subjects are of same gender, similar age, or similar ethnic background. The status of the selected subjects can be confirmed by well established, routinely employed methods including but not limited to general physical examination of the individuals and general review of their medical history.

In some cases, the selected group of reference subjects must be of a reasonable size, such that the average level/amount/concentration of RF and/or ACPA in samples obtained from the group can be reasonably regarded as representative of the normal or average level among this population of patients.

Once the predetermined value for the level of RF and/or ACPA is established based on the individual values found in each subject of the selected group, this representative value or level can be considered a cut-off level. In some embodiments, the representative value is based on a specific percentile distribution of a marker (RF or ACPA) in a reference population.

A standard deviation can also be determined during the same process. In some cases, separate cut-off values may be established for separately defined groups having distinct characteristics such as age, gender, or ethnic background.

Methods for obtaining the reference value from the group of subjects selected are well-known in the state of the art (Burtis et al., 2008, Chapter 14, section “Statistical Treatment of Reference Values”). In some cases, a “reference control level” for rheumatoid factor is a cut-off value defined by means of Receiver Operating Characteristic (ROC) analysis. As a skilled person in the art will appreciate, optimal cut-off values can be defined according to a particular application of the method, a target population for the prediction, a balance between specificity and sensitivity, and other requirements.

E. Anti-TNFα Biological Inhibitors

The anti-TNFα biological inhibitors disclosed herein can be administered to a patient with RA in a therapeutically effective amount for treating one or more symptoms associated with RA. The present invention advantageously enables a clinician to practice “personalized medicine” by guiding treatment decisions and informing therapy selection for RA such that a specific therapeutically effective drug treatment can be administered to a subject in need thereof.

The anti-TNFα biological inhibitors can be administered with a suitable pharmaceutical excipient as necessary and can be carried out via any of the accepted modes of administration. Thus, administration can be, for example, intravenous, topical, subcutaneous, transcutaneous, transdermal, intramuscular, oral, buccal, sublingual, gingival, palatal, intra-joint, parenteral, intra-arteriole, intradermal, intraventricular, intracranial, intraperitoneal, intralesional, intranasal, rectal, vaginal, or by inhalation. By “co-administer” it is meant that an anti-TNFα biological inhibitor is administered at the same time, just prior to, or just after the administration of a second drug (e.g., another anti-TNFα biological inhibitor drug, a drug useful for reducing the side-effects of the RA drug, etc.).

A therapeutically effective amount of an anti-TNFα biological inhibitor drug may be administered repeatedly, e.g., at least 2, 3, 4, 5, 6, 7, 8, or more times, or the dose may be administered by continuous infusion. The dose may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for human subjects and other mammals, each unit containing a predetermined quantity of an anti-TNFα biological inhibitor drug calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the anti-TNFα biological inhibitor drug.

Methods for preparing such dosage forms are known to those skilled in the art (see, e.g., Remington's Pharmaceutical Sciences, 18^{th} ed., Mack Publishing Co., Easton, Pa. (1990)). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., Remington's Pharmaceutical Sciences, supra). Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the therapeutically effective dose takes the form of a pill, tablet, or capsule, and thus, the dosage form can contain, along with an anti-TNFα biological inhibitor drug, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. An RA drug can also be formulated into a suppository disposed, for example, in a polyethylene glycol (PEG) carrier.

Liquid dosage forms can be prepared by dissolving or dispersing an anti-TNFα biological inhibitor drug and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. For topical administration, the therapeutically effective dose can be in the form of emulsions, lotions, gels, foams, creams, jellies, solutions, suspensions, ointments, and transdermal patches. For administration by inhalation, an anti-TNFα biological inhibitor drug can be delivered as a dry powder or in liquid form via a nebulizer. For parenteral administration, the therapeutically effective dose can be in the form of sterile injectable solutions and sterile packaged powders. Preferably, injectable solutions are formulated at a pH of from about 4.5 to about 7.5.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

An anti-TNFα biological inhibitor drug can be administered at the initial dosage of from about 0.001 mg/kg to about 1000 mg/kg daily. A daily dose range of from about 0.01 mg/kg to about 500 mg/kg, from about 0.1 mg/kg to about 200 mg/kg, from about 1 mg/kg to about 100 mg/kg, or from about 10 mg/kg to about 50 mg/kg, can be used. The dosages, however, may be varied depending upon the requirements of the individual, the severity of RA symptoms, and the anti-TNFα biological inhibitor drug being employed. For example, dosages can be empirically determined considering the severity of RA symptoms, the stage of RA, and/or the prognosis of RA in an individual. The dose administered to an individual, in the context of the present invention, should be sufficient to affect a beneficial therapeutic response in the individual over time. The size of the dose can also be determined by the existence, nature, and extent of any adverse side-effects that accompany the administration of a particular anti-TNFα biological inhibitor drug in an individual. Determination of the proper dosage for a particular situation is within the skill of the practitioner. Generally, treatment is initiated with smaller dosages which are less than the optimum dose of the anti-TNFα biological inhibitor drug. Thereafter, the dosage is increased by small increments until the optimum effect under circumstances is reached. For convenience, the total daily dosage may be divided and administered in portions during the day, if desired.

The therapies described herein can be administered to a patient with RA alone or in combination with other types of therapy for RA. Non-limiting examples of other therapies for treating RA include PDE4 inhibitors, disease-modifying anti-rheumatic drugs (DMARDs), non-steroidal anti-inflammatory drugs (NSAIDs), immunosuppressive drugs, corticosteroids, free bases thereof, pharmaceutically acceptable salts thereof, derivatives thereof, analogs thereof, and combinations thereof. Non-limiting examples of DMARDs include methotrexate (MTX), leflunomide, D-penicillamine, gold salts (e.g., sodium aurothiomalate, auranofin, etc.), minocycline, anti-malarial medications (e.g., chloroquine, hydroxychloroquine, sulfasalazine, etc.), free bases thereof, pharmaceutically acceptable salts thereof, derivatives thereof, analogs thereof, and combinations thereof. Examples of NSAIDs include, but are not limited to, ibuprofen, indomethacin, COX-2 inhibitors (e.g., Celecoxib), free bases thereof, pharmaceutically acceptable salts thereof, derivatives thereof, analogs thereof, and combinations thereof. Examples of NSAIDs include, but are not limited to, ibuprofen, indomethacin, COX-2 inhibitors (e.g., Celecoxib), free bases thereof, pharmaceutically acceptable salts thereof, derivatives thereof, analogs thereof, and combinations thereof.

An individual can be monitored at periodic time intervals to assess the efficacy of a certain therapeutic regimen once a course of treatment has been initiated. For example, the presence or level of RF and/or ACPA may change based on the therapeutic effect of a treatment such as an anti-TNFα biological inhibitor drug. In certain embodiments, the patient is monitored to assess clinical response and understand the effects of certain anti-TNFα biological inhibitor drugs or treatments in an individualized approach. In certain other embodiments, patients may not respond to a drug, but the presence or level of RF and/or ACPA, indicating that these patients belong to a special population (not responsive) that can be identified by their autoantibody levels. These patients can be discontinued on their current therapy and alternative treatments prescribed.

### IV. EXAMPLE

The present invention will be described in greater detail by way of a specific example. The following example is offered for illustrative purposes, and is not intended to limit the invention in any manner. Those of skill in the art will readily recognize a variety of noncritical parameters which can be changed or modified to yield essentially the same results.

### Example 1

### Predicting Response to an Anti-TNFα Therapy in Patients with Rheumatoid Arthritis.

This example illustrates a method for predicting a response to an anti-TNFα therapy such as adalimumab, infliximab and etanercept in a patient with rheumatoid arthritis. The method includes determining the patient's level of rheumatoid factor (RF) and anti-cyclic citrullinated peptide autoantibody (anti-CCP or ACPA) compared to an established RF cut-off level and an established ACPA cut-off level.

A. Patients and Methods

All patients in the study were diagnosed with having rheumatoid arthritis according to the American College of Rheumatology (ACR) diagnostic criteria. All patients had>2 years of disease progression, were Caucasian with Spanish ancestry (up to 2 generations) and were older than 18 years old. Patients were treated with one of the following anti-TNFα therapies: adalimumab (ADA, HUIMIRA®, AbbVie Inc.), infliximab (IFX, REMICADE®, Janssen Biotech, Inc.), and etanercept (ETN, ENBREL®, Amgen).

A total of 289 RA patients who responded to anti-TNF treatment at week 12 were used for the present analysis. The cohort included a total of 80.2% women, which is close to the expected 3:1 ratio for disease prevalence.

Whole blood samples were obtained for each patient using BD Vacutainer extraction tubes (Becton Dickinson) with EDTA anticoagulant agent. After 24 hrs at room temperature, tubes were centrifuged and the supernatant (i.e., plasma) was separated and stored at −80° C. until use. The plasma samples from all patients were obtained.

All plasma samples were simultaneously analyzed for anti-CCP titers and RF titers. For anti-CCP quantitation the cobas® anti-CCP in vitro assay (Roche Diagnostics) was used. For the RF quantitation the cobas® Rheumatoid Factors in vitro assay (Roche Diagnostics) was used. The raw data was obtained at the Vall d′Hebron Hospital and submitted to the GRR-VHIR for analysis.

Levels of anti-CCP autoantibodies and rheumatoid factor autoantibodies were determined in all the plasma samples. The results are as follows: 79.6% of the patients were anti-CCP positive; 76.8% of the patients were RF positive; 88.6% of the patients were either anti-CCP positive or RF positive; and 67.8% of the patients were both anti-CCP positive and RF positive.

Treatment response was defined according to the EULAR response criteria. Based on disease activity at week 0 (baseline) and at week 12 of anti-TNF treatment, patients are categorized as “good,” “moderate” or “none” EULAR responders. The cohort was categorized as follows: 32.5% (n=94) were EULAR “good” responders, 40.1% (n-116) were EULAR “moderate” responders; and 27.3% (n-79) were EULAR “none” responders.

Disease activity was measured using the DAS28 score (Disease Activity Score defined by EULAR). Both basal DAS28 as well as DAS28 (i.e., basal DAS28-weekl2 DAS28) were normally distributed variables, according to Shapiro-Wilk's test.

The study was performed according to the patient's initial response to an anti-TNFα treatment. The patient distribution across the three anti-TNFα treatments was as follows: 26.2% of the patients (n=74) responded to adalimumab (ADA); 34.7% of the patients (n=98) responded to etanercept (ETN); and 39% of the patients (n=110) responded to infliximab (IFX).

Patients were then categorized into a binary response: the “good” and “moderate” EULAR responders were grouped together into the “responder” group (72.7% of patients), while the “none” EULAR responders were categorized as the “non-responder” group (Table 1).

TABLE 1
Percentage of Non-responders vs. Responders to
ADA, ETN or IFX

| Response | ADA | ETN | IFX |
|---|---|---|---|
| Non-responder | 21.3 | 23.5 | 33.6 |
| Responder | 78.7 | 76.5 | 66.4 |

The percentage of “non-responders” in the IFX group was higher (P<0.05) than the other 2 treatment groups. This higher percentage of non-responders receiving IFX might be due to the fact that IFX was the first biological therapy to be approved for RA. It is possible that some of these patients had a longer standing disease and also a reduced response to the drug.

All statistical analyses were performed using the R statistical language system (v.2.10.1). Significance between group categorizations was determined using Fisher's exact test.

The concentration (level, amount or titers) of RF and ACPA were conventionally measured using international units per milliliter (IU/mL or U/mL). Using healthy individuals and individuals with RA, a negative to positive cut-off (i.e. absence vs. presence of autoantibody) was estimated.

B. Biomarker Profiles for Predicting Response to anti-TNFα Biological Inhibitor Therapy

1. Investigating Possible Associations Between Therapy Response and the Standard Positive/Negative Classification Analysis for RF and ACPA

Next, the study focused on investigating a possible association between anti-TNFα therapy response and the standard binary positive vs. negative cut-off levels for RF and anti-CCP titers. A quantitative trait analysis was performed using linear regression. For standard methods using Roche's cobas® systems, the established positive cut-offs are >=14 IU/mL for RF-positive patients and >=17 IU/mL for ACPA-positive patients. Using the standard established cut-off, the following correlations were made.

RF level and GLOBAL (ADA, IFX, and ETN): There is no association of all anti-TNFs with response (P=0.21).

RF level and ADA: No significant association (P=0.12).

RF level and ETN: No significant association (P=0.22).

RF level and IFX: There is a significant association with non-response to therapy (P=0.0033).

Anti-CCP level and GLOBAL (ADA, IFX, and ETN): No significant association (P=0.63).

Anti-CCP level and ETN: No significant association (P=0.66).

Anti-CCP level and IFX: No significant association (P=0.74).

Anti-CCP level and ADA: No significant association (P=0.20).

The results show that positive levels of RF (i.e., levels higher than the established binary cut-off value) were associated with non-response in IFX-treated patients. RF levels were not associated with a response in patients treated with ADA or ETN. Furthermore, using the standard binary cut-offs (positive or negative), levels of anti-CCP were not predictive of response or non-response in either ETN-, IFX- or ADA-treated patients. This study shows that standard autoantibody cut-off values failed to correlate with response or non-response to specific anti-TNFα biological inhibitors.

2. Investigating the Association between Therapy Response and Novel High and Low Cut-Off Levels for RF and ACPA

Next, novel optimal autoantibody cut-off values were investigated by screening for a statistically significant association with treatment response (Fisher's test) over different titer thresholds for RF and anti-CCP (ACPA). It was hypothesized that categorizing RA patients into RF high vs. RF low and ACPA high vs. ACPA low could be predictive of anti-TNFα response. Importantly, we hypothesized that a single threshold value for RF and a single threshold value for ACPA could be used for different types of anti-TNFα therapy (monoclonal antibody-based anti-TNFα therapies, e.g., adalimumab and infliximab, and TNF receptor-based biological inhibitors, e.g., etanercept). To find the optimal “high” vs. “low” autoantibody titer to successfully categorize RA patients as responders or non-responders, we scanned all possible autoantibody titer cutoffs, starting from the negative to positive cut-offs and up to the highest autoantibody titers. Significance values were recorded and plotted to identify the most discriminating threshold.

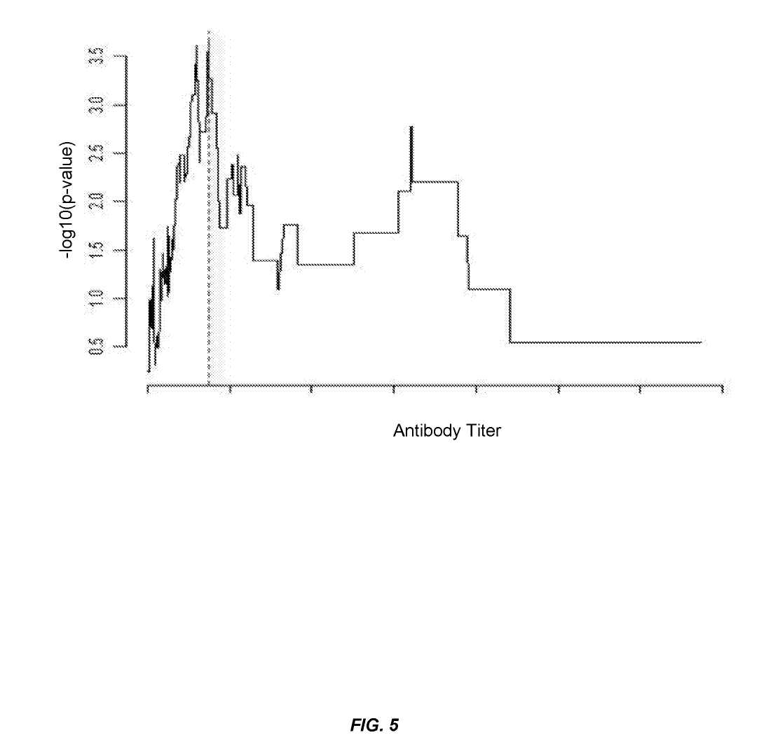

For example we screened for an association between a positive response to a monoclonal antibody-based anti-TNFα therapy and an optimal cut-off level for RF. For each candidate RF cut-off value (candidate RF titer), the RA patients were categorized as either having a high titer of RF or a low titer, depending on whether the patient had an RF value higher than (or equal to) the candidate RF cut-off or an RF value lower than the candidate cut-off, respectively. Next, the patients were also grouped according to their response at week 14 to adalimumab (ADA) or infliximab (IFX) (i.e., “responder” vs. “non-responder”). To determine the optimal RF cut-off value for predicting a positive response to ADA or IFX, the range of proposed RF values was plotted on the x-axis and the significance of an association with a positive response was plotted on the y-axis. The significant of the association or the p-value was also −log 10 transformed to represent a positive and more intuitive visualization. From all evaluated candidate cutoff values, the optimal P-value was −log 10(P-value)=3.64, which corresponds to an RF level of 148 IU/mL (dotted vertical line of FIG. 5). The graph indicates that patients with an RF-high level (e.g., higher than or equal to 148 IU/mL) are predicted to have a poor response or no response to adalimumab (ADA), infliximab (IFX) and the like, while patients with an RF-low level (e.g., lower than 148 IU/mL) are predicted to have a high likelihood of positively responding to such monoclonal antibody-based anti-TNFα therapies.

Also, patients with an RF-high level are predicted to have a high likelihood of responding to a different type of TNFα inhibitor therapy such as etanercept (ETN) or certolizumab pegol (CZP).

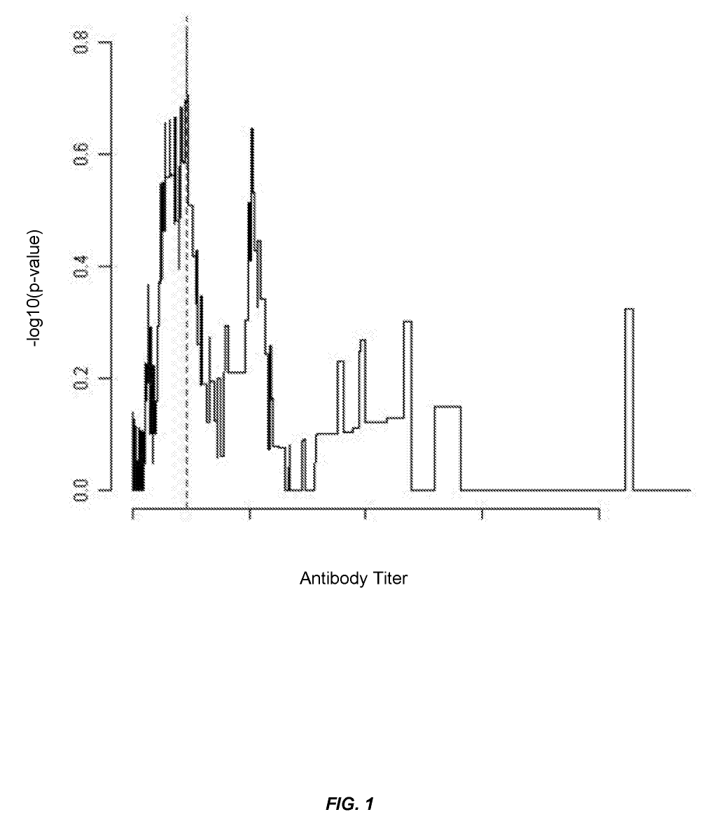

The methods described above were also used to investigate whether a response to an anti-TNFα biological inhibitor can be predicted based on an optimal cut-off value for anti-CCP autoantibodies (ACPAs). As can be seen in FIG. 1, no significant antibody threshold was found when all treatments are analyzed together. However, when performing the screening method for each treatment separately, an apparent correlation was detected.

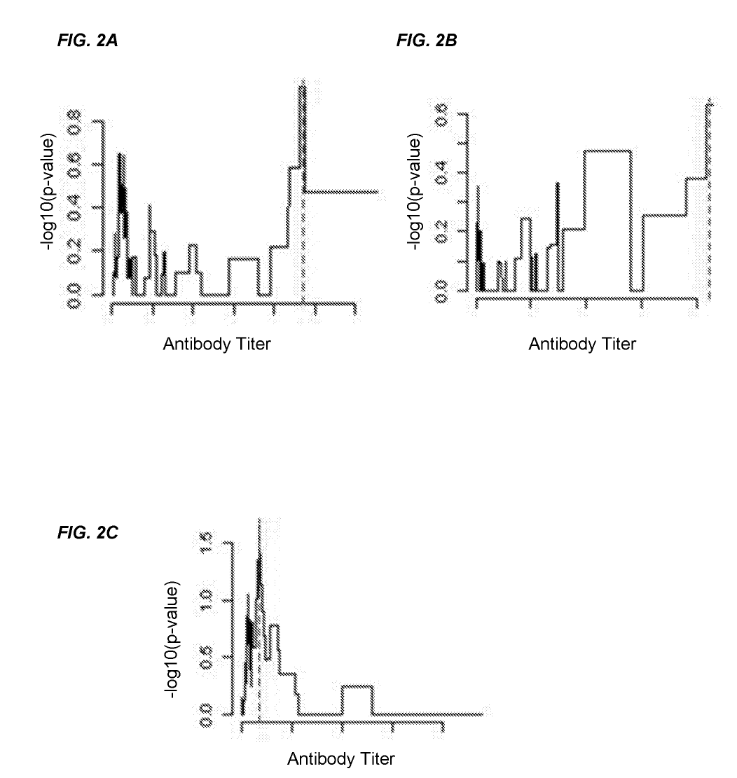

As can be seen in FIG. 2, a specific threshold level of anti-CCP (ACPA) was associated with a positive response to adalimumab (ADA) in ADA-treated patients (P=0.022). 92% of patients with high levels of anti-CCP (e.g., higher than 730 IU/mL) responded to ADA therapy compared to only 70% of patients with low levels of anti-CCP. Patients with low levels of anti-CCP also included the anti-CCP negative patients. A significant association (P=0.05) was also seen at an anti-CCP (ACPA) threshold level that falls within a range of from about 725 IU/mL to about 754 IU/mL. It should be noted that by using the standard negative/positive classification analysis, anti-CCP levels showed no significant association to response to ADA treatment (P=0.56).

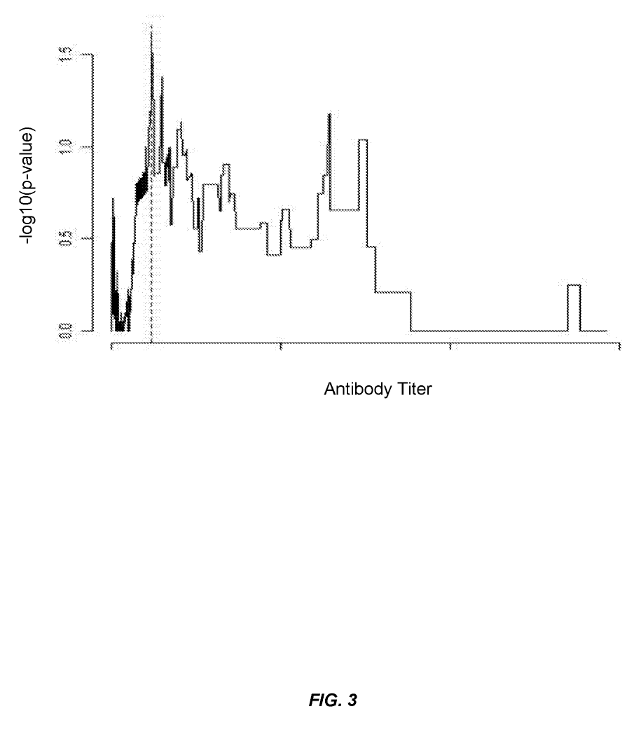

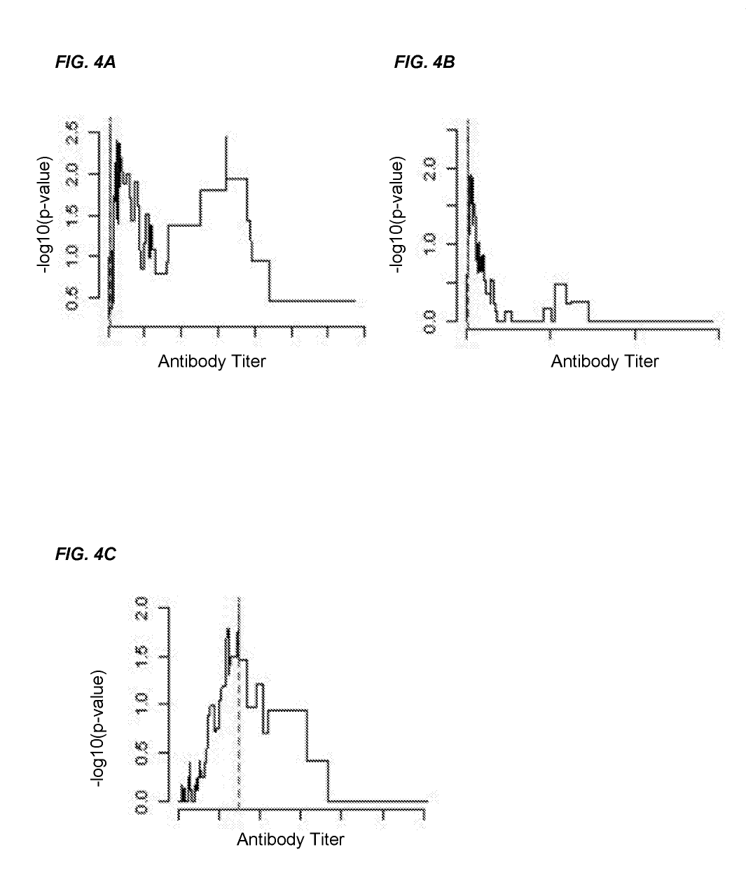

A similar analysis was performed to investigate the association between ADA response and RF levels. As can be seen in FIG. 3, patients at a specific autoantibody threshold could be categorized as responders (P=0.02). We also investigated the association within each anti-TNFα treatment. As seen in FIG. 4, in this treatment subtype analysis we found more significant results. For infliximab (IFX) the association was highly significant (P=0.002) with 40% of RA patients with high levels of RF not responding to treatment compared to 10% patients in the low level group.

For etanercept (ETN) the association was also highly significant (P=0.002) but, importantly, in the inverse sense compared to IFX. Only 15% of RA patients with high RF levels did not respond to ETN compared to 50% patients with low levels.

ADA had a similar behavior to IFX. Non-response or poor response to ADA was associated with high levels of RF (P=0.009). In particular, 62% of patients with high levels of RF failed to respond to treatment, compared to 16% of patients with low levels who failed to respond.

Given that both IFX and ADA showed a similar relationship to RF levels, we evaluated both monoclonal antibody-type treatments together. As seen in FIG. 5, the association with this combined group showed the highest significance of all analyses (P=0.00023). In this combination, 61% of RF-high patients (patients with high levels of RF) did not respond to either IFX or ADA therapy, compared to only 23% of patients with low RF levels. Therefore, patients having high RF levels are predicted to positively respond to anti-TNFα therapies such as etanercept (ETN), certolizumab pegol (CZP) and the like.

The results point to RF and anti-CCP (ACPA) as key predictive variables for predicting response to different classes of anti-TNFα biological inhibitor therapies. Patients who have a high RF level (14% of all) are predicted to have a poor response to a monoclonal antibody-based therapy such as ADA and IFX compared to patients who have low RF levels (86%, P=0.00023). The data as depicted in FIG. 5 shows a statistically significant association between monoclonal antibody-based anti-TNFα therapy response and an RF cut-off level that falls within a range of from about 107 IU/mL to about 159 IU/mL (P<0.001). A significant association of P<0.005 was also found at an RF cut-off level that falls within a range of from about 98 IU/mL to about 172 IU/mL.

The methods and cut-off levels described herein can be generalized for any technology platform used to measure RF and ACPA titers. Although international units (IU) should be equivalent independent of the technology used, there could be variations, e.g., instrumental or chemical variations that could yield slight differences from the methods used in the study described above. In order to adapt the method to other detection platforms, the cut-off values provided herein can be transformed to their equivalent sample quantiles. This is based on the assumption that the RF and ACPA distributions measured using different technologies will be proportional to those of the present study. For example, an RF titer of 148 IU/mL corresponds to the 81^{st} percentile (81%ile) of RF distribution in RA patients. Thus, even if an alternative technology measures RF using a different scale, the cut-off point may represent the 81⁴ percentile of the RF distribution in RA patients. Thus, the cut-off points and ranges disclosed above can be represented as specific percentiles of distribution. For instance, an RF cut-off value of 148 IU/mL corresponds to the 81⁴ percentile of RF distribution in RA patients receiving an anti-TNFα biological inhibitor and an RF threshold range of 107 IU/mL to 159 IU/mL corresponds to the 75^{th}-83^{rd} percentile (75%ile-83%ile) of RF distribution in RA patients. Similarly, the 73^{rd}-85^{th} percentile (73%ile-85%ile) of RF distribution is substantially equivalent to an RF threshold range of 98 IU/mL to 172 IU/mL. An ACPA threshold value of 730 IU/mL corresponds to the 66^{th} percentile (66%ile) of ACPA distribution in RA patients receiving an anti-TNFα biological inhibitor and an ACPA threshold range of 725 IU/mL to 754 IU/mL is substantially equivalent to the 65.8^{th}-66.9^{th} (65.8%ile-66.9%ile) percentile of ACPA distribution.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. An in vitro method for predicting whether a subject with rheumatoid arthritis is likely to respond to treatment with an anti-TNFα biological inhibitor, the method comprising:
(a) detecting a level of rheumatoid factor (RF) in a sample obtained from the subject; and
(b) determining that the subject is not likely to respond to a monoclonal antibody-based anti-TNFα biological inhibitor, but is likely to respond to an antibody fragment-based anti-TNFα biological inhibitor or a TNF receptor-based biological inhibitor, if the level of RF is higher than an RF reference control level.

2. The method of claim 1, wherein if the level of RF is lower than the RF reference control level, the method further comprises:
(c) detecting a level of anti-cyclic citrullinated peptide autoantibody (ACPA) in a sample obtained from the subject; and
(d) determining that the subject is not likely to respond to the antibody fragment-based anti-TNFα biological inhibitor or the TNF receptor-based biological inhibitor, but is likely to respond to the monoclonal antibody-based anti-TNFα biological inhibitor, if the level of ACPA is higher than an ACPA reference control level.

3. The method of claim 2, further comprising determining that the subject is not likely to respond to the monoclonal antibody-based anti-TNFα biological inhibitor, but is likely to respond to the antibody fragment-based anti-TNFα biological inhibitor or the TNF receptor-based biological inhibitor, if the level of ACPA is lower than the ACPA reference control level.

4. An in vitro method for predicting whether a subject with rheumatoid arthritis is likely to respond to treatment with an anti-TNFα biological inhibitor, the method comprising:
(a) detecting a level of anti-cyclic citrullinated peptide autoantibody (ACPA) in a sample obtained from the subject; and
(b) determining that the subject is likely to respond to a monoclonal antibody-based anti-TNFα biological inhibitor, but is not likely to respond to an antibody fragment-based anti-TNFα biological inhibitor or a TNF receptor-based biological inhibitor, if the level of ACPA is higher than an ACPA reference control level.

5. The method of claim 4, wherein if the level of ACPA is lower than the ACPA reference control level, the subject is not likely to respond to a monoclonal antibody-based anti-TNFα biological inhibitor, but is likely to respond to an antibody fragment-based anti-TNFα biological inhibitor or a TNF receptor-based biological inhibitor.

6. The method of claim 5, further comprising:
(c) detecting a level of rheumatoid factor (RF) in the sample; and
(d) determining that the subject is not likely to respond to a monoclonal antibody-based anti-TNFα biological inhibitor, but is likely to respond to an antibody fragment-based anti-TNFα biological inhibitor or a TNF receptor-based biological inhibitor, if the level of RF is higher than an RF reference control level.

7. The method of claim 6, further comprising determining that the subject is not likely to respond to the antibody fragment-based anti-TNFα biological inhibitor or the TNF receptor-based biological inhibitor, but is likely to respond to the monoclonal antibody-based anti-TNFα biological inhibitor, if the level of RF is lower than the RF reference control level.

8. The method of claim 1, wherein the monoclonal antibody-based anti-TNFα biological inhibitor is selected from the group consisting of adalimumab (HUMIRA™), infliximab (REMICADE™), golimumab (SIMPONI®), a biosimilar thereof, and a combination thereof.

9. The method of claim 1, wherein the antibody fragment-based anti-TNFα biological inhibitor is selected from the group consisting of certolizumab pegol (CIMZIA®), a biosimilar thereof, and a combination thereof.

10. The method of claim 1, wherein the TNF receptor-based biological inhibitor is selected from the group consisting of etanercept (ENBREL™), pegsunercept, a biosimilar thereof, and a combination thereof.

11. The method of claim 1, wherein the sample is selected from the group consisting of whole blood, plasma, serum, synovial fluid, saliva, and urine.

12. The method of claim 1, wherein the RF reference control level is an RF level within a range of from about 98 IU/mL to about 172 IU/mL.

13. The method of claim 12, wherein the RF reference control level is an RF level within a range of from about 107 IU/mL to about 159 IU/mL.

14. The method of claim 1, wherein the RF reference control level corresponds to an RF level within about the 73^{rd} percentile (73%ile) to about the 85^{th} percentile (85%ile) of the RF distribution in a control group.

15. The method of claim 2, wherein the ACPA reference control level is an ACPA level within a range of from about 725 IU/mL to about 754 IU/mL.

16. The method of claim 2, wherein the ACPA reference control level corresponds to an ACPA level within about the 65.8^{th} percentile (65.8%ile) to about the 67^{th} percentile (67%ile) of the ACPA distribution in a control group.

17-32. (canceled)

33. A method of treating a human subject having rheumatoid arthritis (RA) comprising administering a therapeutically effective amount of a monoclonal antibody-based anti-TNFα biological inhibitor to a human subject suffering from RA and having a level of rheumatoid factor (RF) lower than an RF reference control level and a level of anti-cyclic citrullinated peptide autoantibody (ACPA) higher than an ACPA reference control level.

34. A method of treating a human subject having rheumatoid arthritis (RA) comprising administering a therapeutically effective amount of an antibody fragment-based anti-TNFα biological inhibitor or a TNF receptor-based biological inhibitor to a human subject suffering from RA and having a level of rheumatoid factor (RF) lower than an RF reference control level and a level of anti-cyclic citrullinated peptide autoantibody (ACPA) lower than an ACPA reference control level.

35. The method of claim 33, wherein the RF reference control level is an RF level within a range of from about 98 IU/mL to about 172 IU/mL.

36. (canceled)

37. (canceled)

38. The method of claim 33, wherein the ACPA reference control level is an ACPA level within a range of from about 725 IU/mL to about 754 IU/mL.

39. (canceled)
